(19) United States
(12) Patent Application Publication
(10) Pub. No.: US 2003/0078557 A1
(43) Pub. Date: Apr. 24, 2003

(54) Absorbent article having double vertical cuff assembly

(76) Inventor: Joseph B. Vergona, Suwanee, GA (US)

Correspondence Address: Christopher C. Campbell, Esq., Hunton & Williams, Suite 1200, 1900 K Street, NW, Washington, DC, 20006-1109, US

(21) Appl. No.: 09/978,577

(22) Filed: Oct. 18, 2001

(51) Int. Cl. A61F 13/15; A61F 13/20

(52) U.S. Cl. 604/385.28; 604/385.29; 604/396

(57) ABSTRACT

An absorbent article having a double vertical cuff assembly comprises a first set of vertical cuffs attached to the surface of a liquid permeable topsheet and generally positioned on top of the absorbent core. The topsheet is preferably wrapped around an absorbent core forming a core assembly. The vertical cuffs attached to the topsheet extend upward from the respective side edges of the core assembly to define a first region there between for the containment of body exudates. The core assembly is positioned on the center of a liquid impermeable backsheet between a second set of vertical cuffs. The space between the second set of vertical cuffs and the core assembly define a second containment region for body exudates.

### BACKGROUND OF THE INVENTION

1. Field of the Invention

The invention relates to the field of absorbent garments in general, and more particularly to an absorbent article having a double vertical cuff assembly for improved containment of bowel movement (BM) leakage.

2. Description of Related Art

Disposable absorbent articles such as infant diapers or training pants, adult incontinence products, and other such products are well-known in the art. Typically, such articles comprise a liquid-permeable body-contacting liner sheet (or “top sheet”), a liquid-impermeable backing sheet (or “back sheet”), and a moisture-absorbent core (or “absorbent core”) that is generally disposed between the liquid-permeable liner sheet and the liquid-impermeable backing sheet. These disposable absorbent articles oftentimes include additional features such as elastic waist bands, elastic leg bands, and stretchable side panels. In addition, decorative graphics are often incorporated into the disposable absorbent article for aesthetic and functional purposes.

Despite advances in the field of absorbent articles, the escape of urinary and fecal excretions from absorbent articles continues to present a formidable challenge to absorbent article manufacturers and others skilled in the art. Leakage of BM and urine from absorbent articles occurs when the absorbent cores of such garments cannot absorb body exudates fast enough, resulting in a lateral flow of the excreted matter toward the sides of the garment. Manufacturers rely on the leg elastic gathers (used to secure the garment to the legs of the wearer) to prevent body exudates from leaking out. The use of leg elastic gathers as containment means is often ineffective, particularly in the case of subsequent insults which take longer to be absorbed due to a partially saturated absorbent core.

Standing leg cuffs or leg gathers were added to disposable absorbent garments to compliment the leg elastic gathers. The standing leg gathers were typically positioned laterally inboard of the leg gathers. The standing leg gathers block the lateral flow of fluids and other waste material. Although the standing leg gathers in the prior art are elasticized to assist further in retaining bodily waste, the use of a singular leg gather has not always been effective in restraining the lateral flow of bodily waste. Bodily waste migrating over the standing leg gather is retained by the leg elastics extending along the leg openings. These leg elastics, however, typically did not provide any vertical separation between the back sheet and the legs of the wearer, and in fact, the leg elastics were typically sandwiched between the backsheet and the topsheet. Consequently, to the extent that bodily waste migrated over the top of the standing leg gathers, conventional diapers were susceptible to leakage beyond their last line of defense, the leg elastic gathers, due in part to the lack of vertical separation between the backsheet and the wearer.

In response to these and other attempts to contain the lateral flow of bodily waste, proposals have been made to incorporate double standing vertical cuff assemblies into a single absorbent garment. For example, U.S. Pat. No. 5,167,653 discloses a double vertical cuff assembly on each side of the absorbent garment. Specifically, the double vertical cuff assembly is positioned atop the topsheet along the lateral side edges of the absorbent garment. Because both cuffs in the '653 Patent are positioned adjacent the side edge of the absorbent article, they are less effective in containing the lateral flow of bodily waste than had they been positioned with a greater degree of separation. Other prior art containing vertical double cuff assemblies is similarly deficient.

It is these deficiencies that the invention of the preferred embodiments has overcome.

### SUMMARY OF THE INVENTION

In response to the difficulties encountered in the prior art, a disposable absorbent article is disclosed having a double vertical cuff assembly having a specified geometry for the cuffs.

The absorbent article preferably comprises a first set of vertical cuffs, preferably formed by attaching elastics to non-woven carrier strips. These cuffs are attached to the top surface of a liquid permeable top sheet, with each cuff being positioned just inboard of a respective side edge of the absorbent core.

The first set of vertical cuffs, along with the top sheet, absorbent core, and transfer sheet (if desired) form a core assembly. The cuffs attached to the top sheet extend upward from the respective side edges of the core assembly to define a first region therebetween for containing body exudates.

The core assembly is positioned on the center of a liquid impermeable back sheet. A second set of vertical cuffs are attached to the back sheet on either side of the core assembly. The space between the second set of vertical cuffs and the core assembly comprises a second containment region for body exudates. Together, the liquid impermeable back sheet, the second set of vertical cuffs, and the core assembly positioned between the cuffs comprise a double vertical cuff assembly. The absorbent article may be used as described above, however, as known in the art, a chassis is preferably included having an outer non-woven layer as well as any combination or derivation of known components, depending on whether the absorbent article is to be a diaper, training pant, adult incontinence product, or the like.

One advantage of the invention is the provision of a first set of vertical cuffs attached to the core assembly. These cuffs define a containment region that is useful in containing insults, and when necessary, subsequent insults which generally take longer to be absorbed due to a partially saturated core. By having the first pair of vertical cuffs attached to the topsheet at a position corresponding with the edge of the absorbent core or, alternatively, laterally inboard of the side edges of the absorbent core, the first pair of vertical cuff assemblies advantageously provide a first barrier against the lateral flow of bodily waste. This causes the bodily waste to have additional time to interact with the absorbent core. Furthermore, one of the important properties of an absorbent core is the ability to absorb and retain subsequent doses of urine. Subsequent insults typically require more time to be absorbed within the core due to the partially swollen superabsorbent polymer contained within the core. By positioning the first vertical cuffs above the absorbent core, most or all of the subsequent insults are required to interact initially with the absorbent core. To the extent that any subsequent insults migrate over the top of the first vertical cuffs, the reservoir created between the first vertical cuffs and the second outboard vertical cuffs provides yet another path for acquiring bodily waste into the absorbent core.

Another advantage of the invention is the provision of a second pair of vertical cuffs, with each cuff being mounted to the backsheet on respective sides of the core assembly. A second containment region is defined by the space between the core assembly and the second set of cuffs. This region provides additional protection which may be needed when multiple, explosive insults cannot be absorbed by the absorbent core fast enough, resulting in a lateral flow of BM and urine toward the sides of the garment.

Yet another advantage of the invention is the ability of the absorbent article to be conveniently joined to a chassis containing an outer non-woven layer. As known in the art, the chassis may include any combination or derivation of known components, enabling the absorbent article to be formed into a diaper, training pant, adult incontinence product, or the like.

These and other objects, features and advantages of the invention will be apparent through the detailed description of the preferred embodiments and the drawings attached hereto. It is also to be understood that both the foregoing general description and the following detailed description are exemplary and explanatory and are not restrictive of the scope of the invention.

### BRIEF DESCRIPTION OF THE DRAWINGS

The invention will be described with respect to the accompanying drawings, in which like elements are referenced with like numbers.

FIG. 1 is an exploded sectional view of an absorbent article having a double vertical cuff assembly, according to an embodiment of the invention.

FIG. 2 is a sectional view of an absorbent article having a double vertical cuff assembly, according to an embodiment of the invention.

FIG. 3 is a perspective view of the embodiment illustrated in FIG. 2.

FIG. 4 is an exploded view of a chassis, on which an absorbent article having a double vertical cuff assembly may be placed.

FIG. 5 shows a schematic of a preferred method for making an absorbent article having a double vertical cuff assembly, according to an embodiment of the invention.

### DETAILED DESCRIPTION OF THE PREFERRED EMBODIMENTS

As used herein, the term “absorbent garment” or “absorbent article” refers to garments that absorb and contain exudates, and more specifically, refers to garments which are placed against or in proximity to the body of the wearer to absorb and contain the various exudates discharged from the body. A non-exhaustive list of examples of absorbent articles includes diapers, diaper covers, disposable diapers, training pants, feminine hygiene products, and adult incontinence products. The term “disposable absorbent garment” refers to absorbent garments that are intended to be discarded or partially discarded after a single use (i.e., they are not intended to be laundered or otherwise restored or reused).

The present invention can be used with all of the foregoing classes of absorbent articles, without limitation, whether disposable or otherwise. These classifications are used interchangeably throughout the specification, but are not intended to limit the claimed invention. The invention will be understood to encompass, without limitation, all classes and types of absorbent articles, including those described above.

The term “component” can refer, but is not limited, to designated selected regions, such as edges, corners, sides or the like; structural members, such as elastic strips, absorbent pads, stretchable layers or panels, layers of material, or the like; or a graphic.

The following detailed description will be made in the context of an absorbent article having a double vertical cuff assembly in order to improve the containment of body exudates.

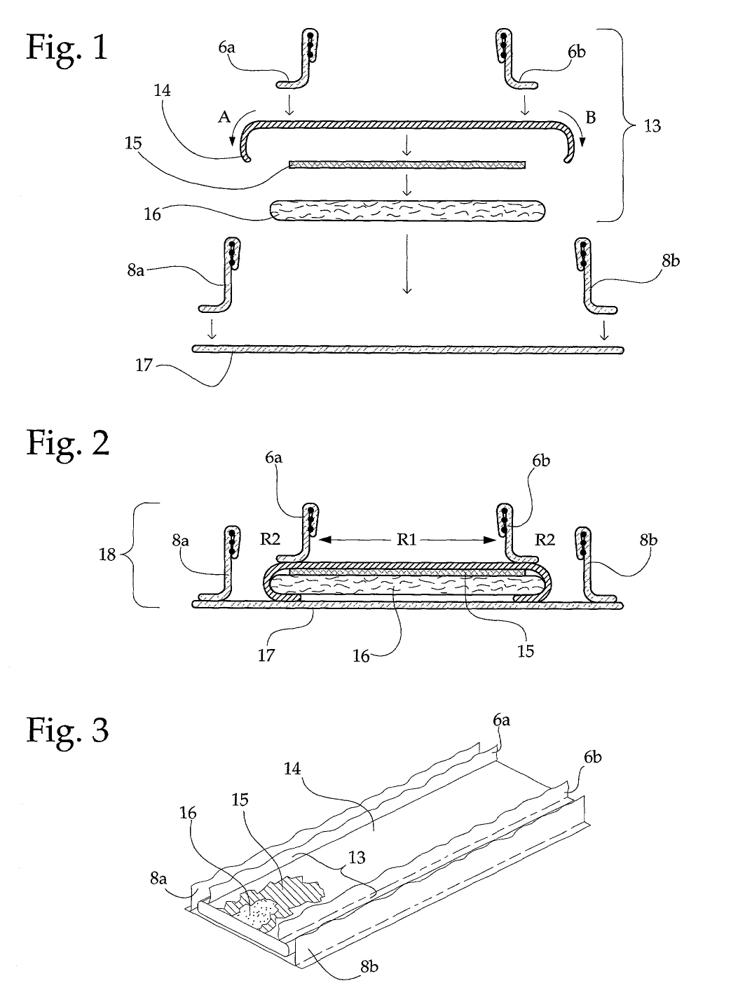

An embodiment of the invention is first described with reference to FIGS. 1-3. An absorbent core 16 is positioned between a liquid impermeable back sheet 17 and a liquid permeable top sheet 14. Absorbent core 16 may include a fluff component, preferably comprising cellulosic fibers, such as comminuted softwood pulp fibers. The fluff component may also be mixed with distributed particles of a superabsorbent polymer (SAP), with the resulting fluff/SAP core being preferably surrounded by a tissue layer over-wrap (not illustrated) or other known absorbent material, in order to contain the SAP. It should be recognized, however, that absorbent core 16 may be comprised of any absorbent material or materials known in the art, and accordingly should not be limited to the description above. Furthermore, the absorbent core 16 may extend the entire length of the disposable absorbent garment, i.e., from the front waist section to the back waist section, but it alternatively may extend only partially along the length of the disposable absorbent garment. The absorbent core 16 may be rectangular along its entire length. Alternatively, the absorbent core 16 may have an hourglass shape or a T-shape with the absorbent core extending into one or more ears of the absorbent garment.

A transfer layer 15 may optionally be included adjacent the absorbent core 16, if so desired. Transfer layer 15 may preferably be comprised of any material having a wicking ability, and may be designed to cover only a portion of the surface of absorbent core 16. Alternatively, it may extend across the entire surface of absorbent core 16, or may even surround absorbent core 16. The transfer layer 15 is particularly adapted to perform its function of transferring fluid from the topsheet to the absorbent core when the first vertical cuffs 6a, 6b are positioned above the absorbent core 16 and preferably at the upper corners thereof. As bodily fluid is discharged, the first pair of vertical cuffs 6a, 6b contain and hold the discharged fluid for a time sufficient to permit the fluid to be captured by the transfer layer until it may be wicked into the absorbent core 16.

Liquid permeable top sheet 14 is preferably made from any suitable material known in the art, including polymeric fabrics such as polyolefin non-woven fabrics. Common polyolefin non-woven fabrics include polypropylene and polyethylene spunbonded fabrics. Additionally, the top sheet may also be formed from non-woven bicomponent polymeric fabrics. The liquid permeable topsheet 14 may be surface treated to render it hydrophilic.

A first set of vertical cuffs (6a, 6b) are joined to the top surface of the liquid permeable top sheet 14, with each cuff being positioned just inboard of a respective side edge of top sheet 14. Vertical cuffs (6a, 6b) are preferably formed by attaching elastics to non-woven carrier strips. They may be joined to top sheet 14 using any known adhesives, or by using known ultrasonic or thermal bonding techniques. Vertical cuffs (6a, 6b) preferably extend the entire length of top sheet 14. The first set of vertical cuffs 6a, 6b preferably include one or more and most preferably a plurality of elastics encapsulated within a portion of the non-woven carrier strips folded onto themselves. The elastics preferably extend parallel to one another within the folded envelope of the vertical cuffs and may exhibit different degrees of extensibility depending upon their location within the vertical cuffs. For instance, the elastics closest the absorbent core may exhibit a higher degree of elasticity than the elastics further from the absorbent core. The converse construction is also within the scope of the preferred embodiments.

As illustrated in FIG. 1, the respective side edges of top sheet 14 are preferably wrapped around absorbent core 16 in the direction illustrated by arrows A and B. It should be recognized however that top sheet 14 may simply be attached to the top surface of the absorbent core. Together, top sheet 14, transfer layer 15 (if present), and absorbent core 16 form a core assembly 13 having a single set of vertical cuffs (6a, 6b). Vertical cuffs (6a, 6b) extend upward from the respective side edges of core assembly 13, as seen in FIG. 2, to define a first region R1 therebetween for containing body exudates. Having a first containment region R1 is advantageous, especially in the event of subsequent insults which take longer to be absorbed due to a partially saturated absorbent core.

Core assembly 13 is shown positioned on a liquid impermeable back sheet 17. Back sheet 17 is preferably formed from polyethylene, however any suitable material can be used, as is known in the art. Core assembly 13 is positioned on the center of back sheet 17, and preferably extends along the entire length of back sheet 17. The back sheet is preferably wide enough to accommodate a second set of vertical cuffs (8a, 8b) joined to the top surface thereof. Each cuff of the second set of vertical cuffs (8a, 8b) is joined to a respective side edge of back sheet 17, as is shown in FIG. 2. The second set of vertical cuffs (8a, 8b) are also preferably formed by attaching elastics to non-woven carrier strips, and are joined to back sheet 17 using suitable known adhesives, or by ultrasonic or thermal bonding techniques. As with the first vertical cuff assembly, the second set of vertical cuffs (8a, 8b) may include elastics positioned within an envelope formed by folding the non-woven carrier strips onto itself. Furthermore, the elastics, which are preferably parallel to one another along the length of the vertical cuffs (8a, 8b), may exhibit different degrees of elasticity depending on their location within the folded envelope.

Together, liquid impermeable back sheet 17, the second set of vertical cuffs (8a, 8b), and core assembly 13 positioned between the cuffs, comprise a double vertical cuff assembly 18. A second region R2 for containing body exudates is formed between cuffs (8a, 8b) and core assembly 13. This region provides additional containment means which may be necessary in the event of a lateral flow of excreted matter. Lateral flow may result when multiple, explosive insults cannot be absorbed by the cores of disposable garments fast enough, causing the excreted matter to flow toward the sides of the garment.

FIG. 3 is a partially revealed perspective view of the embodiments of the absorbent article shown in FIGS. 1 and 2. In FIG. 3, the core assembly 13 is shown extending the entire length of back sheet 17 and having its ends flush with the respective ends of back sheet 17. In alternative embodiments (not illustrated), core assembly 13 may extend along only a portion of the length of back sheet 17 with its ends positioned inboard from the respective ends of back sheet 17. Additionally, the first set of vertical cuffs (6a, 6b) may extend only partially along the length of core assembly 13 in various embodiments, depending on how they are attached to top sheet 14. Likewise, the second set of vertical cuffs (8a, 8b) may be mounted inboard from the respective side edges of back sheet 17, and may only extend partially along the length of back sheet 17.

As previously stated, the absorbent article having a double vertical cuff assembly may be disposed on a chassis. The chassis may include any combination or derivation of known components, effectively allowing the absorbent article to be formed into a number of final products that include, but are not limited to, diapers, diaper covers, disposable diapers, training pants, feminine hygiene products, and adult incontinence products.

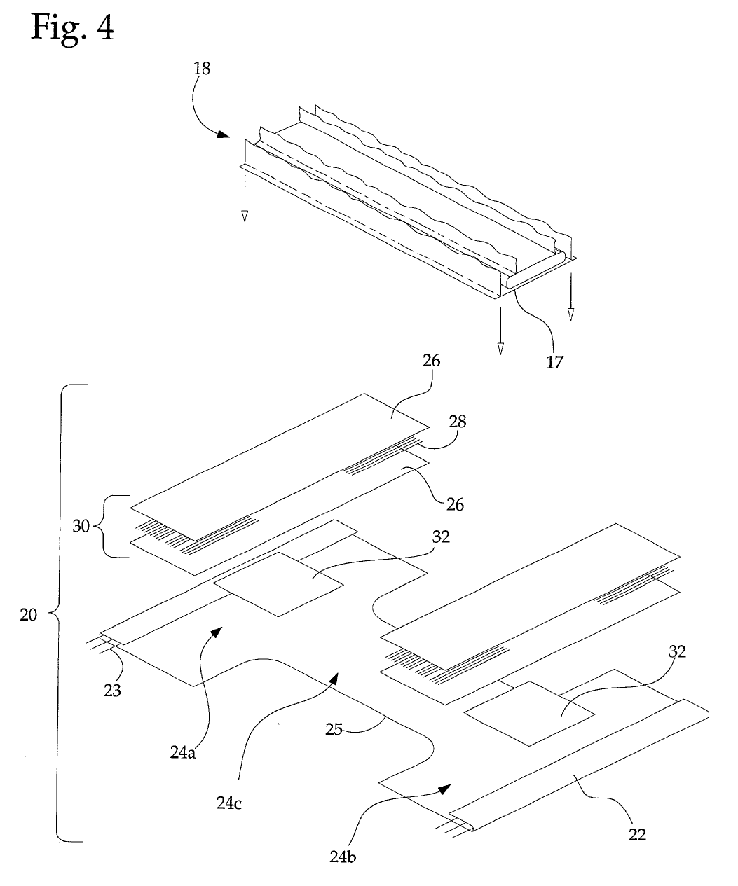

As an example, FIG. 4 illustrates the absorbent article with a double vertical cuff being placed on to a chassis 20 in order to form a training pant. The chassis 20 includes a layer of outer non-woven material 22 that is preferably formed from a non-woven fabric having a cloth-like texture. The outer nonwoven 22 has a front waist portion 24a, a rear waist portion 24b, and a crotch portion 24c. Elastic waist elements (tummy elastics) 23 are shown extending across the top edges of the front and rear waist portions (24a, 24b) of outer non-woven 22. In addition, elastic leg elements (not shown) may extend along leg openings 25 of outer non-woven 22. The chassis 20 may further include, for example, elastic side panels 30 that are positioned between the liquid impermeable back sheet 17 of the double vertical cuff assembly 18, and the outer non-woven layer 22 in order to provide elasticity thereto.

The outermost non-woven layer 22 may preferably include at least one graphic 32 positioned thereon. The graphic 32 generally includes a visually pleasing design or pattern and is applied at a preferred, designated location on either the exterior or interior face of non-woven layer 22. Typically, one graphic may be positioned on the front waist portion 24a of non-woven layer 22 while another corresponding graphic is positioned on rear waist portion 24b.

The elastic side panels 30 preferably comprise a composite of elastic elements 28 and carrier strips 26. During production, the elastic elements 28 extend entirely across the width of the waist opening. However, the elastic elements 28 are cut, causing them to snap back to the side edges of the article corresponding to the area where the elastics have been adhesively attached to the carrier strips 26. In this manner, the elastic elements 28 are positioned so as not to overlap the graphics 32. Again, the foregoing description of the absorbent article used in a training pant is exemplary only and not intended to limit the scope of the invention.

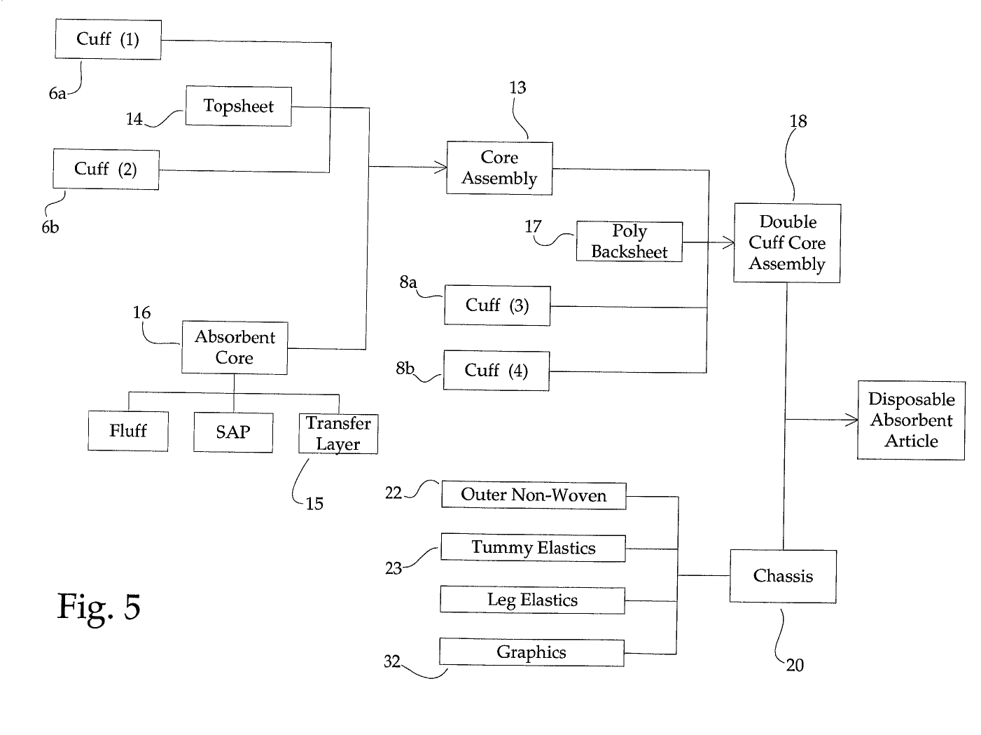

A disposable absorbent article having a double vertical cuff assembly may preferably be produced according to the system schematically illustrated in the flow diagram of FIG. 5. It should be recognized that the absorbent article and chassis are produced by assembling the necessary various components in a continuously moving production line (or process), as is well-known in the art. Accordingly, any known, suitable method of manufacture may be used.

Generally stated, a first set of vertical cuffs (6a, 6b) is formed by attaching elastics to non-woven carrier strips. The first set of cuffs (6a, 6b) are then joined to the top surface of a liquid permeable top sheet 14, either off-line or in the production line, using known adhesives, or ultrasonic or thermal bonding techniques. Each cuff is positioned slightly inboard of (and extending the entire length of) a respective side edge of top sheet 14. The top sheet, which may be made from any suitable polymeric fabric, is fed into the process and is preferably wrapped around an absorbent core 16. Absorbent core 16 may, as known in the art, comprise a combination of cellulosic fibers (fluff) and particles of a superabsorbent polymer (SAP). A transfer layer 15 constructed from a material having high-wicking properties may also be included either on or around the absorbent core 16, although not required.

The top sheet 14, cuffs (6a, 6b), absorbent core 16, and transfer layer 15 (if present) together comprise a core assembly 13. Core assembly 13 is positioned on a back sheet 17, preferably comprised of polyethylene. A second set of vertical cuffs (8a, 8b) are also fed into the process and attached to back sheet 17, with one cuff being placed on either side of core assembly 13. Together, back sheet 17, vertical cuffs (8a, 8b), and core assembly 13 positioned between the cuffs comprise a double vertical cuff assembly 18.

Other methods may be employed to manufacture an article of the present invention, and various steps of the process disclosed herein may be modified, replaced, or reordered without leaving the scope of the invention.

The absorbent article may be used as described above, however, as known in the art, it is preferably positioned on a chassis 20 that may include any combination or derivation of known components, depending on whether the absorbent article is to be a diaper, training pant, adult incontinence product, or the like. The chassis, for example, may include an outer non-woven 22, as well as tummy elastics 23, leg elastics, or graphics 32.

As discussed previously, the double vertical cuff assembly of the preferred embodiments advantageously provide two impediments to the lateral flow of bodily waste. These impediments, unlike the prior art, are sufficiently spaced from one another to create a first absorption region positioned exclusively above the absorbent core and a second absorption region positioned between the respective inner and outer vertical cuffs. Furthermore, by virtue of having a double vertical cuff assembly, to the extent that bodily waste migrates over the inner vertical cuffs, separation between the wearer and the backsheet is maintained by virtue of the outer vertical cuffs. This provides an additional containment region not available in prior art absorbent garments.

Further benefits may be obtained by making the first set of vertical cuffs (6a, 6b) and the second set of vertical cuffs (8a, 8b) from a liquid impervious material. In previously disclosed garments having multiple vertical cuffs, it has been the case that the inner set of vertical cuffs has been manufactured from a liquid permeable material. Such a construction is disclosed, for example, in U.S. Statutory Invention Registration H1,630 issued to Roe et al., which is incorporated herein by reference in its entirety. A preferred embodiment of the present invention may be provided with a liquid impervious first set of vertical cuffs (6a, 6b). In such an embodiment, fluid entering the first region R1 may be contained between the first set of vertical cuffs (6a, 6b) so that it may be distributed longitudinally along the garment, rather than laterally through the first set of vertical cuffs (6z, 6b), to fully utilize the absorbent core 16 from end to end. When the first region R1 becomes overloaded, fluids may pass between the first set of cuffs (6a, 6b) and the wearer's body to be captured between the first set of vertical cuffs (6z, 6b) and the second set of vertical cuffs (8a, 8b) in the second region R2. Fluid in the second region R2 may then be absorbed into the edges of the absorbent core 16 along the core's entire length, thereby fully utilizing the capacity of the absorbent core 16.

Other benefits may be obtained in an embodiment of the invention constructed with a liquid impervious first set of vertical cuffs (6a, 6b). Liquid pervious cuffs may transfer fluid to the wearer's skin during use, causing discomfort to the wearer. For example, when a wearer sits down, the first set of vertical cuffs (6a, 6b) may collapse, and fluid from a wet absorbent core 12 may migrate through the collapsed cuffs and to the wearer's skin. The liquid impervious first set of cuffs (6a, 6b) may serve as a barrier to help prevent fluid in the core from contacting the wearer's skin when the cuffs are collapsed.

Other embodiments, uses and advantages of the invention will be apparent to those skilled in the art from consideration of the specification and practice of the invention disclosed herein. The specification should be considered exemplary only, and the scope of the invention is accordingly intended to be limited only to the following claims.

We claim:

1. An absorbent article, comprising:
a top sheet;
a back sheet;
an absorbent core disposed between at least a portion of said top sheet and at least a portion of said back sheet, wherein a first pair of vertical cuffs are mounted on the topsheet opposite one another along respective side edges of said absorbent core, said top sheet and said absorbent core forming a core assembly, wherein said first pair of vertical cuffs extend upward from respective side edges of said core assembly to define a first region there-between for containing body exudates; and
a second pair of vertical cuffs mounted on said back sheet on opposite sides of said core assembly and laterally outboard of said first pair of vertical cuffs, creating a second region between said core assembly and said second pair of vertical cuffs for containing body exudates.

2. The absorbent article of claim 1, further comprising a chassis on which said back sheet is mounted.

3. The absorbent article of claim 1, wherein said top sheet is partially wrapped around said absorbent core.

4. The absorbent article of claim 1, wherein said top sheet is made from a liquid permeable non-woven material.

5. The absorbent article of claim 1, wherein said back sheet is made from a liquid impermeable material.

6. The absorbent article of claim 5, wherein said liquid impermeable material is polyethylene.

7. The absorbent article of claim 1, further comprising a transfer layer disposed between at least a portion of said top sheet and at least a portion of said absorbent core.

8. The absorbent article of claim 1, wherein said core assembly is centered on said back sheet, and extends the entire length of said back sheet.

9. The absorbent article of claim 1, wherein said first pair of vertical cuffs extend along the entire length of said core assembly.

10. The absorbent article of claim 1, wherein said first pair of vertical cuffs extend only partially along the length of said core assembly.

11. The absorbent article of claim 1, wherein said second pair of vertical cuffs extend along the entire length of said back sheet.

12. The absorbent article of claim 1, wherein said first pair of vertical cuffs are made from a liquid impermeable material.

13. The absorbent article of claim 1, wherein said second pair of vertical cuffs are made from a liquid impermeable material.

14. An absorbent article, comprising:
a top sheet;
a back sheet;
an absorbent core disposed between at least a portion of said top sheet and at least a portion of said back sheet, wherein a first pair of vertical cuffs are mounted on the topsheet opposite one another along respective side edges of said top sheet and on top of said absorbent core, said top sheet being partially wrapped around said absorbent core forming a core assembly, wherein said first pair of vertical cuffs extend upward from respective side edges of said core assembly to define a first region there-between for containing body exudates;
a second pair of vertical cuffs mounted on said back sheet on opposite sides of said core assembly, creating a second region between said core assembly and said second pair of vertical cuffs for containing body exudates.
